(19) United States
(12) Patent Application Publication
(10) Pub. No.: US 2008/0124343 A1
(43) Pub. Date: May 29, 2008

(54) Treatment of Inappropriate Immune Responses

(76) Inventors: He Xu, Rockville, MD (US); Allan D. Kirk, Atlanta, GA (US)

Correspondence Address: SCHWEGMAN, LUNDBERG & WOESSNER/NIH, PO BOX 2938, MINNEAPOLIS, MN, 55402-0938, US

(21) Appl. No.: 11/629,934

(22) PCT Filed: Jun. 16, 2005
(86) PCT No.: PCT/US05/21364

§ 371 Date: Dec. 10, 2007

Related U.S. Application Data
(60) Provisional application No. 60/581,201, filed on Jun. 18, 2004.

(51) Int. Cl. A61K 39/395; A61K 39/00; A61P 43/00; C07K 16/00

(52) U.S. Cl. 424/172.1; 424/184.1; 530/387.1; 530/387.3

(57) ABSTRACT

The invention relates to compositions containing antibodies that can bind activated lymphocytes and/or activated monocytes. The invention also provides methods for making these antibodies and methods for using these antibodies to treat or prevent undesirable immune responses.

This application claims benefit of the filing date of U.S. Provisional Application Ser. No. 60/581,201, filed Jun. 18, 2004, the contents of which are incorporated herein in their entirety.

### GOVERNMENT FUNDING

The invention described herein was developed with support from the National Institutes of Health. The U.S. Government has certain rights in the invention.

### FIELD OF THE INVENTION

The invention relates to treatment of inappropriate immune responses, for example, autoimmune diseases, graft versus host diseases, allograft rejection, multiple sclerosis, diabetes and the like.

### BACKGROUND OF THE INVENTION

Activated human leukocytes play an essential role in counter-adaptive immune responses such as allograft rejection, autoimmune diseases, and graft-versus-host disease. Depletion of leukocytes involved in these responses by using preparations of leukocyte-specific antibodies may be therapeutic in preventing and reversing these conditions. To date, however, the available monoclonal preparations do not have sufficiently broad specificity to limit the activity of many types of cells involved in counter-adaptive immunity, and the available polyclonal preparations have significant side effects caused by their unintended specificity for bystander cells or cells with beneficial properties.

Accordingly, more selective antibody preparations and methods for blocking inappropriate immune responses are needed.

### SUMMARY OF THE INVENTION

The invention provides polyclonal antibodies that can be used to treat and prevent undesirable immune responses, as well as antigenic formulations and methods for generating those polyclonal antibodies. The polyclonal antibodies of the invention specifically target activated immune cells that have known detrimental effects. The polyclonal antibodies of the invention have few unintended depletional or blocking effects upon bystander cells not involved in generating an inappropriate immune response. Hence, the polyclonal antibodies of the invention can be used to treat inappropriate immune responses with improved specificity compared to currently available antibody preparations.

Another aspect of the invention is an antigenic formulation that includes activated monocytes and activated lymphocytes. This antigenic formulation can be used to create antibody preparations of the invention. In some embodiments, the activated monocytes and activated lymphocytes are human activated monocytes and human activated lymphocytes. Such an antigenic formulation is useful for preparing polyclonal antibodies that can bind to activated immune cells while avoiding bystander cells that are not involved in detrimental or undesirable immune responses. Thus, the polyclonal antibodies of the invention can be used to treat and prevent undesirable immune responses.

Another aspect of the invention is a method of treating an undesirable immune response in a mammal comprising administering to the mammal an antibody preparation that can bind to activated mammalian lymphocytes and activated mammalian monocytes. In some embodiments, the activated mammalian lymphocytes are human activated lymphocytes and/or the activated mammalian monocytes are human activated monocytes. The antibody preparation can include antibodies that have a substantially human antibody sequence especially, for example, when the mammal to be treated is a human.

In some embodiments, the undesirable immune response involves rejection of transplanted cells or tissues, autoimmune disease, arthritis, an inflammatory bowel disease, an endocrinopathy, a neurodegenerative disease, or a vascular disease. In other embodiments, the undesirable immune response is rejection of allogeneic cells, tissues or organs, rejection f xenogeneic cells, tissues or organs, graft versus host disease, systemic or discoid lupus erythematosus, sclerosing cholangitis, autoimmune hepatitis, rheumatoid arthritis, psoriasis, psoriatic arthritis, ulcerative colitis, Crohn's disease, type 1 diabetes, Graves disease, multiple sclerosis, autistic spectrum disorder, Alzheimer's disease, amyotrophic lateral sclerosis (ALS), Parkinson's disease, Huntington's Disease, Guillain-Barre syndrome, myasthenia gravis, chronic idiopathic demyelinating disease (CID), autoimmune hearing loss, systemic vasculitis, or atherosclerosis.

Another aspect of the invention is a method for generating polyclonal antibodies that can bind to activated immune cells. In general, the method involves immunizing a suitable host animal with in vitro activated T-cells and cytokine activated monocytes. Preferred host animals have been genetically modified to generate human antibodies. Purified immunoglobulin from these host animals produces a polyclonal preparation with heightened specificity for activated leukocytes and decreased activity for resting or beneficial leukocytes as well as other blood elements. The preparation is enriched for antibodies targeting molecules that are up-regulated following leukocyte activation. This method can be used to produce large amounts of therapeutic immunoglobulin for infusion into individuals at risk for or suffering from allograft rejection, autoimmune disease, graft versus host disease or other counter-adaptive leukocyte mediated diseases.

### DESCRIPTION OF THE FIGURES

FIG. 1 illustrates the effect of polyclonal antibodies prepared according to the methods of the invention on the in vitro proliferative capacity of lymphocytes responding to allogeneic lymphocytes in a mixed lymphocyte culture. Small, clinically relevant amounts of immunoglobulins that were isolated from a rabbit immunized with both activated monocytes and activated lymphocytes (rabbit 2, at 5 μg) inhibit cellular proliferation of cells in a mixed lymphocyte reaction, reducing proliferation to the level of the negative control (“vs. self”). The low dose blocking effect is most pronounced when using antibodies derived from a rabbit immunized with both activated monocytes and activated lymphocytes according to the methods of the invention. When tested at higher doses (e.g. 50 μg), polyclonal antibodies isolated from a rabbit immunized with activated monocytes and lymphocytes (Rabbit 2) or resting monocytes and lymphocytes (Rabbit 1) can stimulate lymphocytes beyond that seen in the positive control (“vs. donor”), perhaps through binding to a cell surface marker such as CD3. These responses are compared to three controls. The positive control is the response of cells against allogeneic targets without antibody treatment and is labeled the “vs. donor” control. The negative control is the response without any allogenic cells (irradiated autologous cells were used to simulate responders) and is labeled the “vs. self” control. The specificity control involved treatment with antibody derived from rabbits that have not been immunized (labeled “isotype Ig”).

FIG. 2A-D illustrate that certain types of immune cells were depleted from blood samples treated with polyclonal antibodies isolated from rabbits immunized with activated monocytes and activated lymphocytes (rabbit 2) or resting leukocytes (rabbit 1) and that the most efficient depletion is achieved using activated cells prepared according to the invention. The controls used included untreated cells (Normal) and cell treated with antibodies from rabbits that were not immunized (Isotype Ig).

FIG. 2A shows that polyclonal antibodies from a rabbit immunized with both activated human monocytes and activated human lymphocytes (rabbit 2) were more effective for depleting blood samples of white blood cells (WBC) than were polyclonal antibodies from a rabbit immunized with resting human leukocytes (rabbit 1).

FIG. 2B shows that polyclonal antibodies from either a rabbit immunized with both activated human monocytes and activated human lymphocytes (rabbit 2) or from a rabbit immunized with resting human leukocytes (rabbit 1) were effective at depleting blood samples of lymphocytes.

FIG. 2C shows that polyclonal antibodies from a rabbit immunized with both activated human monocytes and activated human lymphocytes (rabbit 2) were more effective for depleting blood samples of monocytes than were polyclonal antibodies from a rabbit immunized with resting human leukocytes (rabbit 1).

FIG. 2D shows that polyclonal antibodies from either a rabbit immunized with both activated human monocytes and activated human lymphocytes (rabbit 2) or from a rabbit immunized with resting human leukocytes (rabbit 1) only modestly depleted blood samples of platelets (a beneficial cell) and that the level of depletion did not reach clinically problematic levels (<100,000).

FIG. 3A-C illustrate that T lymphocytes stimulated with PMA and ionomycin are activated prior to their use as immunogens for rabbits. These T lymphocytes express CD25 (FIG. 3A), CD154 (FIG. 3B) and CD69 (FIG. 3C). These up-regulated markers are desirable targets for antibodies treating T cell mediated diseases. Human PMBC were activated with PMA and ionomycin, then subjected to FACS analysis after staining for the T cell-specific marker, CD3 (as an indicator of T lymphocytes, x-axis), and CD25, CD154 and CD69 (FIGS. 3A, B and C, respectively; y-axis).

FIG. 4A-C illustrate that monocytes stimulated with gamma-interferon are activated prior to their use as immunogens for rabbits in that they express CD40 (FIG. 4A), CD80 (FIG. 4B) and CD54 (FIG. 4C). These up-regulated markers are desirable targets for antibodies useful for treating monocyte-mediated diseases. Human PMBC were activated with gamma-interferon, then subjected to FACS analysis after staining for the monocyte-specific marker, CD14 (x-axis), and CD40, CD80 and CD54 (FIGS. 4A, B and C, respectively; y-axis).

FIG. 5 shows that rabbit anti-activated leukocyte polyclonal immunoglobulin (RALG) blocked endothelial-derived CD54 expression on activated human endothelial cells, as detected by FACS analysis. Isolated IgG from non-immunized animals exhibited no such inhibition of CD54 expression when compared to endothelial cells that were not exposed antibodies. CD54 is a desirable target to prevent leukocyte interactions with endothelial cells during inflammatory immune responses.

FIG. 6A-B shows that human lymphocyte proliferation stimulated by allogeneic endothelial cells (EC) is inhibited after treatment with the rabbit anti-human leukocyte polyclonal antibodies (RALG). Lymphocytes are stimulated to proliferate by allogeneic human EC when no antibodies are present (see bar labeled “w/o Ab”). When lymphocytes were incubated with isolated IgG from non-immunized animals (isotype Ig), no such inhibition of lymphocyte cell proliferation was observed. Unstimulated lymphocytes exhibit low levels of proliferation. FIG. 6A illustrates the effects of 10 μg/ml RALG while FIG. 6B shows the effects of 1 μg/ml RALG upon human lymphocyte proliferation. The effect is present even at the very low dose of 1 μg/ml

FIG. 7A-B also shows that human lymphocyte proliferation in response to xenogeneic cells is inhibited after treatment with the rabbit anti-human leukocyte polyclonal antibodies (RALG). In this experiment lymphocytes were stimulated to proliferate by xenogeneic porcine endothelial cells (EC). Porcine cells were chosen because pigs are thought to be suitable sources of cells and organs for xenogeneic transplantation. FIG. 7A shows lymphocyte cell proliferation after incubation with varying amounts of isolated IgG from non-immunized animals. No inhibition of lymphocyte cell proliferation was observed. However, when lymphocytes were incubated with varying amounts of RALG up to 20 μg/ml, as shown in FIG. 7B, lymphocyte cell proliferation is dramatically inhibited When no antibodies are present the xenogeneic endothelial cells stimulate significant lymphocyte proliferation (see bar labeled “w/o Ab”). Unstimulated lymphocytes exhibited low levels of cellular growth. As in the allogeneic experiments, high doses of antibody (50 μg/ml) stimulated lymphocyte proliferation. Thus, treatment with the rabbit anti-human leukocyte polyclonal antibodies at low doses blocks human lymphocyte proliferation to xenogeneic targets.

### DETAILED DESCRIPTION OF THE INVENTION

The invention provides polyclonal antibodies that have utility for treating and preventing undesirable immune responses. The polyclonal antibodies of the invention can be generated using antigenic formulations provided by the invention. Such antigenic formulations include activated monocytes and activated lymphocytes. The polyclonal antibodies of the invention can be used for depleting or inhibiting the function of immune-related cells both in vitro and in vivo.

The antibodies of the invention are therefore useful for treatment of and prophylaxis against diseases such as hyperimmune syndrome, graft-versus-host disease, and host-versus-graft disease, for preventing or treating rejection when transplanting bone marrow, kidneys, hearts, lungs, pancreases, skin, livers, etc., for preventing or treating rejection of cellular grafts such as islets, for preventing or treating tissue rejection after skin or organ transplant, for T-cell dependent allergic and autoimmune diseases (myocarditis, diabetes, myasthenia gravis, lupus erythematosus, Crohn's disease, multiple sclerosis, AIDS, encephalomyelitis, arthritis, etc.), and for interleukin-2 receptor expressing tumor diseases such as T-cell leukemia or other malignancies or leukemias stemming from transformed lymphocytes or monocytes.

### DEFINITIONS

The term “allogeneic” means that tissues, cells or organs are from different individuals of the same species. Two or more individuals are said to be allogeneic to one another when the genes at one or more loci are not identical.

The term “immunoglobulin” or “Ig”, as used herein is defined as a class of plasma proteins, which function as antibodies. Immunoglobulins include IgA, IgG, IgM, IgE, or IgD and/or their subtypes, for example IgG₁, IgG₂, IgG₃, IgG₄, IgA₁, or IgA₂. IgA functions as the primary antibody that is present in body secretions, such as saliva, tears, breast milk, gastrointestinal secretions and mucus secretions of the respiratory and genitourinary tracts. IgG functions as the most common circulating antibody.

The term “pharmaceutically acceptable carrier” as used herein includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the immunoglobulins or antigenic formulations of the present invention, its use in therapeutic compositions is contemplated. Supplementary active ingredients also can be incorporated into the compositions.

The term “specifically bind,” refers to sufficiently high avidity and/or high affinity binding of an antibody to a specific antigen or antigen-expressing entity, in this case to an activated immune cell, to achieve a desired effect such as depletion or function inhibition of the antigen or cell. As provided herein, antibody binding to an activated immune cell type is stronger than binding of the same antibody to other antigens, particularly resting immune cells. Antibodies that specifically bind to activated immune cells may be capable of binding to other cells, including resting immune cells at a weaker, yet detectable, level (e.g., binding of insufficient affinity or avidity, in some cases 50% or less, to mediate therapeutic depletion or inhibition of the resting immune cells compared to that shown when binding to activated immune cells). Such weak binding, or background binding, is readily discernible from the specific binding of the antibody preparation to activated immune cells, for example, by use of appropriate controls. In general, antibodies used in the invention bind to activated immune cells such that, in comparison to resting immune cells, the proliferation and/or functioning of the activated immune cells is inhibited. In some embodiments, the antibodies of the invention have a binding affinity for activated immune cells of about 10⁻⁷ M or about 10⁻⁸ M or more (e.g., 10⁻⁹ M, 10⁻¹⁰, 10⁻¹¹, etc.). In general, an antibody with a binding affinity of 10⁻⁵ M or less is not as useful in that it will not selectively bind to activated immune cells (over resting immune cells) and not modulate the proliferation and function of those activated immune cells to the degree that antibody preparations with greater binding specificity would. In other cases the affinity is not different but the content of the polyclonal antibody preparations derived from immunization with activated cells is such that more of the antibodies bind to antigen(s) present selectively on activated cells than preparations derived from immunization with resting cells. In other cases, the presence of an antigen is higher on activated cells compared to resting cells such that the same amount of antibody is more effective on activated cells than on resting cells.

The term “subject” or “patient” as used herein is taken to mean any mammalian subject to which an immunoglobulin composition is administered according to the methods described herein. In a specific embodiment, the methods of the present invention are employed to treat a human subject or patient.

The term “therapeutically effective time” as used herein refers to a time frame in which the immunoglobulin composition is still active within the subject.

The term “therapeutically effective amount” as used herein refers to an amount that results in an improvement or remediation of the symptoms of the disease or condition.

The term “treating” and “treatment” as used herein refers to administering to a mammal a therapeutically effective amount of an immunoglobulin composition so that the mammal has an improvement in immune-mediated disease or condition. The improvement is any improvement or remediation of the symptoms. The improvement can be an observable or measurable improvement. Thus, one of skill in the art realizes that a treatment may improve the disease condition, but may not be a complete cure for the disease.

The term “xenogeneic” means that tissues, cells or organs are from individuals of different species.

### Activation of Lymphocytes and Monocytes

The invention involves generating polyclonal antibodies against activated lymphocytes and monocytes. The lymphocytes employed can include activated T-cells or T-lymphocytes. Accordingly, the invention provides an antigenic formulation that includes a carrier and a mixture of activated lymphocytes and monocytes. Such an antigenic formulation is useful for preparing the polyclonal antibodies of the invention.

Purified lymphocytes and monocytes can be obtained from peripheral mononuclear blood cells by any available procedure. For example, peripheral mononuclear blood cells (PBMCs) can be isolated from fresh blood. Peripheral blood can be collected from healthy individuals into a blood collection bag containing an anti-clotting agent, and then centrifuged (e.g., at 600×G, at room temperature for 5 minutes) to separate a blood cell fraction from blood plasma.

The blood cell fraction (excluding blood plasma) can then be diluted with phosphate buffered saline (PBS), layered on Ficoll-Paque, and then applied to density gradient centrifugation (e.g., 400×G, at room temperature for 30 minutes), so as to separate mononuclear cells. The erythrocytes contained together with the mononuclear cells can be hemolyzed by treatment with an ammonium chloride buffer (0.83% NH₄Cl-Tris HCl 20 mM, pH 6.8) at room temperature for 2 minutes, and then the mononuclear cells can be washed with PBS containing about 5% fetal calf serum (PBS-FCS) or some other serum supplement including human serum, or serum-free media. This cell population can be used as peripheral blood mononuclear cells of healthy individuals. See, e.g., Zucker-Franklin et al, Proc. Natl. Acad. Sci. USA 71:1877-1881 (1974).

Further purification of lymphocytes and monocytes can be obtained by using monoclonal antibodies against cell surface markers present on various cell types, including lymphocytes and monocytes. This additional purification is desirable to limit the specificity of the immunized animal's immune response to those cells that lead to pathological conditions, for example, to activated T cells and monocytes. Thus, for example, B cells can be removed by using antibodies directed against the B cell-specific marker, CD19. PMBCs, peripheral blood, or preparations of lymphocytes and monocytes can be incubated with antibody preparations and cells reacting with selected antibodies can be discarded or retained as desired by one of skill in the art. For example, antibodies directed against the B cell-specific marker, CD19, or the marker for antigen presenting cells, CD54 can be used, and cells to which the antibodies bind can be removed by cell sorting, by passage through a column with a matrix that binds the antibodies or by other means available to one of skill in the art. The presence of markers such as CD19, HLA class II-DR (anti-IgG antibody) or CD54 (an adhesion molecule) indicates that B cells and antigen presenting cells may still be present in the preparation.

T cells can be recognized and retained by using antibodies that recognize the T cell-specific marker, CD3. A mixture of granulocyte and monocytes can be detected by using antibodies against a monocyte-specific marker, CD15. Monocytes can be distinguished from other cells by using the monocyte-specific marker, CD14.

Lymphocytes and monocytes can be activated by a variety of agents including foreign antigens, monoclonal antibodies and lectins such as phytohemaglutinin and concanavalin A. Many plant substances, known collectively as lectins or phytomitogens, such as phorbol 12-myristate 13-acetate (PMA), phytohaemagglutinin (PHA), concanavalin (ConA), and lipopolyssacharide have been shown activate lymphocytes.

For example, agents that can be used to activate lymphocytes and monocytes include phorbol 12-myristate 13-acetate (PMA), phytohaemagglutinin (PHA), Concanavalin (ConA), lipopolyssacharides (e.g. from bacteria), anti-CD3/CD28 coated beads, interferon-γ, interleukin-2 and the like. Human lymphocytes can also be activated by PMA in combination with ionomycin in vitro. For example, human peripheral blood mononuclear cells can be stimulated with PMA at a concentration of about 3 to about 30 ng/ml and ionomycin at a concentration of about 80 to about 400 ng/ml.

Monocytes can be activated with cytokines such as interferons. In some embodiments, the monocytes are activated with interferon-gamma (IFN-γ). For example, human monocytes can be incubated with recombinant human IFN-γ at a concentration of about 20 ng/ml to about 200 ng/ml for about 16 hours.

Activation of cells can be monitored by observing whether activation or maturation markers are expressed by the cells. One indication of T cell activation is induction of proliferation. Other indicators of activation include increased lymphokine production and cytotoxic cell activity. T cell activation can be monitored by the up-regulation of cell surface markers such as CD25 (IL-2 receptor), CD69, CD40, CD86 and CD154, for example, as detected by flow cytometry analysis. Activation of monocytes can be monitored by the up-regulation of CD40, CD86 and CD80, for example, as detected by flow cytometry analysis. The presence of markers such as CD45RO, a marker associated with memory T cells, indicates that T cells are present and have been activated. In some embodiments, the generation of memory T cells for inoculation would be desirable, and the presence of memory cells can be detected or monitored by their expression of CD45RO, CD62L and CD95. Enhanced expression of HLA-A, HLA-B and HLA-C (class I antigens), relative to resting cells, is also an indication that the cells, particularly monocytes, are activated. Expression of HLA-DR relative to resting cells is also an indication that both T cells and monocytes are activated.

### Antibody Production

Polyclonal antibodies against activated lymphocytes and activated monocytes can be produced in a variety of animals. The term “antibody” as used herein is defined as a serum immunoglobulin that has specific binding sites to combine with antigens. All antibodies have a similar overall structure and are known collectively as immunoglobulins. Thus, as used herein, the terms “antibody” and “immunoglobulin” are interchangeable.

Polyclonal antibodies of the invention can be raised using procedures available in the art. For example, a suitable mammalian host can be immunized with activated lymphocytes and monocytes in a schedule that includes an initial immunization as well as subsequent immunizations. Initial immunizations are carried out, for example, by intradermal injections of cell suspensions of activated lymphocyte and monocytes (an antigenic formulation of the invention). The amount of activated lymphocytes and monocytes can vary, for example, about 1×10⁶ activated lymphocytes and 1×10⁹ activated monocytes can be used for an initial immunization. In one embodiment, a mixture of about 1×10⁸ activated lymphocytes and 1×10⁸ activated monocytes were used for initial immunization. The activated lymphocytes and monocytes can be formulated with a suitable adjuvant before immunization. For example, the antigenic formulation that includes activated lymphocytes and monocytes can be emulsified with complete Freund's adjuvant (1:1/volume). The total volume of the first immunization can vary. In some embodiments, the first immunization is no greater than about one ml. Intradermal injections can be performed at multiple sites.

Immunized host animals can be given subsequent booster immunizations by a similar or a different route. For example, for the booster immunizations, the cells can be suspended in 5 ml sterile normal saline and infused intravenously into the host animal. A mixture of about 1×10⁶ to 1×10⁹ activated lymphocytes and activated monocytes can be used for subsequent immunization. In one embodiment, about 50-100×10⁶ activated lymphocyte and monocytes were used for the subsequent boosts in a rabbit host. The number of subsequent immunizations can vary. For example, about one to about ten subsequent immunizations can be used. In one embodiment, rabbit hosts received a second immunization 21 days after the first immunization, then the rabbits received immunizations at 28, 29, 30, 35 and 56 days after the first immunization.

Antibodies can be collected from the host animals as the antibodies are produced, during a time period ranging from a week or two after the first immunization to several months after the first immunization. For example, in one embodiment antibodies were collected from rabbit hosts about 35 days and at about 56 days after the first immunization.

Polyclonal antibodies, raised against a mixture of activated human lymphocytes and activated human monocytes in rabbits, were deposited under the terms of the Budapest Treaty on May 21, 2004 with the American Type Culture Collection (10801 University Blvd., Manassas, Va., 20110-2209 USA (ATCC)) as ATCC Accession No. ATCC Number PTA-6009.

The invention contemplates administering the antibody or immunoglobulin preparations of the invention to any animal in need thereof. Thus, the antibodies or immunoglobulins of the invention can be administered to animals such as horses, goats, cattle, sheep, chickens, turkeys, rats, mice, cats, dogs and humans. In some embodiments, the antibodies or immunoglobulins are preferably administered to humans.

In general, one of skill in the art may prefer to use immunoglobulins or antibodies that have constant region sequences substantially identical to the immunoglobulins or antibodies typically produced by the subject mammal to be treated. Thus, “human” immunoglobulins or human antibodies are preferably used in humans; horse immunoglobulins or horse antibodies are preferably used in horses; dog immunoglobulins or dog antibodies are preferably used in dogs, etc.

When the antibodies or immunoglobulins of the invention are administered to humans, human immunoglobulins or humans antibodies are preferably used if long term administration is contemplated. The term “human immunoglobulin” or “human antibody” refers to an immunoglobulin comprising a human framework, in which any constant region present is substantially identical to a human immunoglobulin constant region. In some instances, the human immunoglobulin or human antibody can include a few non-human residues in the human framework region. Thus, for example, the human immunoglobulin or human antibody can be at least about 60% to about 90%, or at least about 95% identical to a human immunoglobulin framework. Moreover, as described herein, the immunoglobulin or antibody need not be raised in a human. Hence, the immunoglobulin or antibody can have at least one CDR from a non-human host. Hence, all parts of a human immunoglobulin or antibody, except possibly the CDRs, are substantially identical to corresponding parts of one or more native human immunoglobulin sequences.

Non-human antibodies can be used in humans for short periods of time. Hence, some inappropriate or undesirable immune responses can be treated with non-human antibodies, particularly when the immune response is short-lived. For example, inappropriate or undesirable immune responses in a human that can be treated with non-human antibodies for short periods of time include tissue rejection, transplant rejection, graft vs. host disease, allograft rejection, and the like. However, such non-human antibodies may give rise to an immune response when used to treat human patients for longer than a few weeks.

For long term usage in human patients, human antibodies are preferred. Thus, human antibodies are preferred when treating diseases such as asthma, bronchitis, lung inflammation, osteoarthritis, juvenile arthritis, rheumatoid arthritis, spondylo arthropathies, gouty arthritis, chronic granulomatous diseases such as sarcoidosis, and silicosis, nephritis, amyloidosis, ankylosing spondylitis, chronic bronchitis, scleroderma, systemic lupus erythematosus, polymyositis, inflammatory bowel disease, Crohn's disease, gastritis, irritable bowel syndrome, ulcerative colitis and for the prevention of Sjogren's syndrome, Reiter's syndrome, psoriasis, orbital inflammatory disease, thrombotic disease, cystitis, tendinitis, bursitis, psoriasis, eczema, burns, dermatitis and inappropriate allergic responses to environmental stimuli such as poison ivy, pollen, insect stings and certain foods, including atopic dermatitis and contact dermatitis. The antibodies of the invention are also useful for treating inflammation in vascular diseases, periarteritis nodosa, thyroiditis, scleroderma, type I diabetes, myasthenia gravis, sarcoidosis, nephrotic syndrome, Behcet's syndrome, polymyositis, gingivitis, hypersensitivity, conjunctivitis, swelling occurring after injury, myocardial ischemia, and the like.

In order to produce human antibodies, the antibodies of the invention can be produced in animals that have been genetically altered to produce human antibodies. Such animals have been generated or are being generated by a number of researchers. For example, cows have been genetically altered to produce human antibodies. See Robl et al., U.S. Patent Application Publication No. 20040068760, entitled Transgenic ungulates capable of human antibody production (Apr. 8, 2004); Kuroiwa et al., Cloned Transchroinosomic Calves Producing Human Immunoglobulin, Nature Biotechnology vol. 20 (September 2002); Cloned Cows Produce Human Antibodies, NewScientist.com News Service (Aug. 12, 2002). Human antibodies from such genetically altered cattle can be obtained commercially from Hematech LLC (Westport, Conn.). Other animals have been genetically altered to produce human antibodies. See Kucherlapati et al., U.S. Pat. No. 6,713,610, entitled Human antibodies derived from immunized xenomice (issued Mar. 30, 2004).

Thus, in some embodiments, a transgenic domestic animal (e.g. a rabbit, sheep, pig, cow) comprising at least a portion of human light and heavy chain immunoglobulin loci can be used. The transgenic domestic animal can be generated by replacement of the animal's immunoglobulin genetic loci with human immunoglobulin genes or by stepwise modification of the animal by gene conversion.

Moreover, animal host cells can be genetically modified to include human immunoglobulin genes. Such animal host cells include fibroblasts, keratinocytes, myocytes, hepatocytes, epithelial cells, or other cells which may be grown and expanded in culture and do not have a rearranged genome. These host cells are transformed (genetically modified) by the introduction of DNA fragments into the cells, where the fragments become integrated into the host genome. Introduction may be by a variety of methods, including bare DNA, transfection with a viral vector, fusion, biolistics, liposomes, etc. The particular method will be selected to accommodate introduction of the large or small amounts of DNA needed and the efficiency of integration. Functional immunoglobulin light and heavy chain loci can be modified by homologous recombination, by replacing at least a portion of the host heavy chain constant region with at least a functional portion of the human heavy chain constant region and if desired, analogously, the host light chain constant region with a human light chain constant region. Of particular interest is also the replacement of the V region most proximal to the D region with a human V region element.

In this way, while some portions of the immunoglobulin are host sequences, the anti-sera are not likely to cause a strong immune response in view of the great variety of variable regions in the anti-sera. In animals, where antibody diversity is generated predominantly by gene conversion, replacement of the V region most proximal to the D region with a human V region element results in expression of the human V element in the majority of immunoglobulins. For the replacement of constant regions it is of particular interest to include at least about 2 of the 3 domains C_{H1}, C_{H2}, and C_{H3}, of the constant region, particularly including C_{H3}.

This genetic engineering is followed by breeding hosts of the same species and selecting for a host that is capable of responding to immunization with production of substantially human anti-sera including host glycosylation, where the immunoglobulin has at least a functional portion of the human heavy chain. Animals expressing the substantially human protein sequence of immunoglobulins are used for the generation of polyclonal antibody preparations by immunization with activated human lymphocyte (e.g., T-cells) and cytokine activated human monocytes. After purification of the anti-sera, such anti-sera may be used, by itself or in combination, with other reagents for the depletion of immune-related cells or for immunomodulation.

Various animals, particularly domestic animals that can provide reasonable volumes of anti-sera may be employed. The animals generally are at least 1 kg, preferably 2 kg, and may be 5 kg or more when adult, although smaller animals can be used as appropriate. Also the gestation period should be less than 12 months, usually being in the range of 1 to 4 months. Illustrative animals include Lagomorpha, e.g. rabbit, ovine, bovine, canine, feline, equine, and the like. Of particular interest are animals where diversification of the antibody repertoire is accomplished predominantly by gene conversion (i.e. rabbits, pigs, sheep, and cattle). In these animals, replacement of the V region element proximal to the D region with a human V region element will result in the expression of the human V region element in the majority of immunoglobulins.

Antibody preparations are obtained by fractionating blood of genetically engineered animals expressing human sequence immunoglobulins. A concentrated immunoglobulin fraction may be prepared by chromatography (affinity, ionic exchange, gel filtration, etc.), selective precipitation with salts such as ammonium sulfate, organic solvents such as ethanol, or by precipitation with polymers such as polyethylene glycol. Antibody preparations may be separated into specific fractions to include selected isotypes including IgA, IgG, IgM, IgE or IgD and/or their subtypes, for example, IgG₁, IgG₂, IgG₃, IgG₄, IgA₁, or IgA₂.

In most instances the antibody preparation will consist of unmodified immunoglobulins. Alternatively, the immunoglobulin fraction may be subject to treatment such as enzymatic digestion (e.g. with pepsin, papain, plasmin, glycosidases, nucleases, etc.), heating, and/or further fractionation. Moreover, immunoglobulins can be made into powders by conventional freeze-drying (or lyophilization) procedure. One or more stabilizing substances can be added to the immunoglobulin preparation prior to the freeze-drying process. A variety of stabilizing substances are employed including, e.g., amino acids such as glycine and lysine, carbohydrates such as dextrose, mannose, galactose, fructose, lactose, sucrose, maltose, sorbitol, mannitol and the like.

The antibody preparations used for administration are generally characterized by containing a polyclonal antibody population, having immunoglobulin concentrations from 0.1 to 100 mg/ml, more usually from 1 to 10 mg/ml. The antibody preparation may contain immunoglobulins of various isotypes. Alternatively, the antibody preparation may contain antibodies of only one isotype, or a number of selected isotypes.

The antibodies may be dissolved or diluted in non-toxic, non-pyrogenic media suitable for intravenous or subcutaneous administration in humans, for example, in sterile buffered saline. In some applications, antibody preparations may be applied directly onto epithelium or administered subcutaneously. For topical or mucosal applications, fractionated antibodies may be dissolved in a water soluble gel such as KY-jelly and the like.

The antibody preparations are often administered into the vascular system, conveniently intravenously by injection or infusion via a catheter implanted into an appropriate vein. The antibody preparation is administered at an appropriate rate, generally ranging from about 10 minutes to about 24 hours, more commonly from about 30 minutes to about 6 hours, in accordance with the rate at which the liquid can be accepted by the patient. Administration of the effective dosage may occur in a single infusion or in a series of infusions. Repeated infusions may be administered once a day, alternating days, once a week, once a month, or once every three months, depending on the half-life of the antibody preparation, the resiliency of the disease process, and the clinical indication.

In other embodiments, the antibodies are administered subcutaneously.

For applications on epithelial surfaces the antibody preparations are applied to the surface in need of treatment in an amount sufficient to provide the intended end result, and can be repeated as needed.

### Modulating Immune Responses

According to the invention, polyclonal immunoglobulin preparations capable of binding to activated lymphocytes and monocytes can be used to treat undesirable immune responses. Immune-mediated diseases are inflammatory diseases perpetuated by antibodies and cellular immunity. The immune response damages healthy organs either inadvertently as a result of attacking foreign substances that have entered the body or foreign cells, tissues or organs placed in the body for therapeutic purposed (e.g. transplants), or by attacking self tissues that happen to resemble foreign substances, a process called autoimmunity.

These diseases include many forms of rejection (e.g. acute humoral or cellular allograft and xenograft rejection), arthritis (e.g., rheumatoid arthritis and psoriatic arthritis), inflammatory bowel diseases (e.g., ulcerative colitis and Crohn's disease), endocrinopathies (e.g., type 1 diabetes and Graves disease), neurodegenerative diseases (e.g., multiple sclerosis, autistic spectrum disorder, Alzheimer's disease, amyotrophic lateral sclerosis (ALS), Parkinson's disease, Huntington's Disease, Guillain-Barre syndrome, myasthenia gravis, and chronic idiopathic demyelinating disease (CID)), and vascular diseases (e.g., autoimmune hearing loss, systemic vasculitis, and atherosclerosis). These diseases are common and have a major socioeconomic impact.

The immunoglobulin preparations of the invention are useful for binding and neutralizing antigenic entities on or in immune-related cells that elicit undesired or abnormal immune responses. An “antigenic entity” is herein defined to encompass any cell, soluble molecule or cell-surface bound molecule (including a protein, lipid or carbohydrate), that may contribute to the development of an immune response and that is capable of binding to an antibody. Treatment with the antibody preparations of the invention can avoid many of the adverse side effects typically associated with administration of immunosuppression drugs.

The immune system of both humans and animals include two principal classes of lymphocytes: the thymus derived cells (T cells), and the bone marrow derived cells (B cells). Mature T cells emerge from the thymus and circulate between the tissues, lymphatics, and the bloodstream. T cells exhibit immunological specificity and are directly involved in cell-mediated immune responses (such as graft rejection). T cells act against or in response to a variety of foreign structures (antigens). In many instances these foreign antigens are expressed on host cells as a result of infection. However, foreign antigens can also be generated when the host has been altered by neoplasia or infection.

Although T cells do not themselves secrete antibodies, they are usually required for antibody secretion by the second class of lymphocytes, B cells.

There are various subsets of T cells, which are generally defined by antigenic determinants found on their cell surfaces, as well as functional activity and foreign antigen recognition. Some subsets of T cells, such as CD8⁺ cells, mediate direct cytotoxicity and in some cases have regulatory functions, while others, such as CD4⁺ cells, serve to promote inflammatory and humoral responses and also have regulatory function in some cases. CD refers to cell differentiation cluster; the accompanying numbers are provided in accordance with terminology set forth by the International Workshops on Leukocyte Differentiation. Immunology Today, 10:254 (1989). A general reference for all aspects of the immune system may be found in Klein, J. Immunology: The Science of Self-Nonself Discrimination, Wiley & Sons, N.Y. (1982).

T-lymphocytes play a particularly important role in achieving cell-mediated immunity. The induction of an antigen specific T cell response requires multiple interactions between cell surface receptors on a T cell and ligands on an antigen presenting cell. The primary interaction is between the T cell receptor/CD3 complex and a major histocompatibility complex molecule, which presents an antigenic peptide to the T cell receptor, thereby triggering an antigen specific signal in the T cells. In addition to this antigen specific signal, a T cell response requires a second, costimulatory signal. A costimulatory signal can be generated in a T cell by stimulation of the T cell through cell surface receptor CD28 (Harding, F. A. (1992) Nature 356:607-609). Ligands for CD28 have been identified on antigen presenting cells (APCs). CD28 ligands include members of the B7 family of proteins, such as B7-1 (CD80) and B7-2 (CD86) (Freedman, A. S. et al. (1987) J. Immunol. 137:3260-3267; Freeman, G. J. et al. (1989) J. Immunol. 143:2714-2722; Freeman, G. J. et al. (1991) J. Exp. Med. 174:625-631; Freeman, G. J. et al. (1993) Science 262:909-911; Azuma, M. et al. (1993) Nature 366:76-79; Freeman, G. J. et al. (1993) J. Exp. Med. 178:2185-2192). Additionally, B7-1 and B7-2 have been shown to bind another surface receptor on T cells related to CD28 termed CTLA4 (Linsley, P. S. (1991) J. Exp. Med. 174:561-569; Freeman, G. J. et al. (1993) Science 262:909-911). In contrast to CD28 which is constitutively expressed on T cells, CTLA4 is induced on T cells upon activation (Linsley, P. S. et al. (1992) J. Exp. Med. 176:1595-1604). Hence, CTLA4 can be used as a marker for T cell activation.

T cells are not only key regulators of the immune response to infectious agents but are believed to be critical for the initiation and maintenance of the inflammatory reaction in a variety of chronic diseases. Increased numbers or enhanced activation state of T cells, especially CD4+ T cells, have been demonstrated in the synovium of individuals with rheumatoid arthritis (M. J. Elliott and R. N. Maini, Int. Arch. Allergy Immunol. 104: 112-1125, 1994), in the bronchial mucosa of asthmatics (C. J. Corrigan and A. B. Kay, Immunol. Today 13:501-506, 1992), in the lesions of multiple sclerosis patients (R. Martin and H. F. McFarland, Crit. Rev. Clin. Lab. Sci. 32: 121-182, 1995), in psoriatic lesions (J. L. Jones, J. Berth-Jone, A. Fletcher and P. E. Hutchinson, J. Pathol. 174: 77-82, 1994) and in the fatty streaks of atherosclerosis (R. Ross, Annu. Rev. Physiol. 57: 791-804, 1995).

In one embodiment of the invention the immunological cells responsible for undesirable immune responses are blocked from contributing to the immune response or reduced in number within a mammal (e.g. a human) by administration of the immunoglobulin preparations of the invention. Thus, the invention provides a method of treating or preventing an undesirable immune response in a mammal that involves administering an effective amount of a polyclonal antibody preparation, wherein antibodies in the polyclonal antibody preparation can bind to activated lymphocytes and/or activated monocytes but exhibit substantially no binding to cells that are not involved in an immune response. Such treatment reduces the numbers of activated lymphocytes and activated monocytes in the lymphocyte population of the mammal and thereby suppresses undesirable immune responses. This treatment can avoid some of the adverse side effects typically associated with administration of immunosuppression drugs.

Undesirable immune diseases include autoimmune diseases such as rheumatoid arthritis, osteoarthritis, juvenile arthritis, diabetes, tissue rejection, asthma, nephritis, chronic bronchitis, systemic or discoid lupus erythematosus, inflammatory bowel disease, sclerosing cholangitis, autoimmune hepatitis, Crohn's disease, gastritis, irritable bowel syndrome, ulcerative colitis, psoriasis, psoriatic arthritis, orbital inflammatory disease, tendinitis, bursitis, eczema, dermatitis and inappropriate allergic responses to environmental stimuli such as poison ivy, pollen, insect stings and certain foods, including atopic dermatitis and contact dermatitis. The antibody compositions and methods of the invention are also useful for treating undesirable immune responses in vascular diseases, periarteritis nodosa, thyroiditis, scleroderma, myasthenia gravis, nephrotic syndrome, Behcet's syndrome, polymyositis, gingivitis, hypersensitivity, conjunctivitis, swelling occurring after injury, myocardial ischemia, and the like.

The antibodies of the invention accordingly are appropriate for the treatment of and prophylaxis against such diseases as hyperimmune syndrome, graft-versus-host disease, and host-versus-graft disease, for preventing and treating rejection of transplanted bone marrow, kidneys, hearts, lungs, pancreases, skin, livers, etc., for preventing and treating rejection of cellular grafts such as islets, for preventing tissue rejection such as skin and organ transplants, for T-cell dependent allergic and autoimmune diseases (myocarditis, diabetes, myasthenia gravis, lupus erythematosus, Crohn's disease, multiple sclerosis, AIDS, encephalomyelitis, arthritis, etc.), and for interleukin-2 receptor expressing tumor diseases such as T-cell leukemia or other malignancies or leukemias stemming from transformed lymphocytes or monocytes.

Other diseases that can be treated by the polyclonal antibodies of the invention include autoimmune hemolytic anemia (including, but not limited to cryoglobinemia or Coombs positive anemia), autoimmune neonatal thrombocytopenia, idiopathic thrombocytopenia purpura, autoimmunocytopenia, autoimmune neutropenia, hemolytic anemia, antiphospholipid syndrome, dermatitis (e.g. atopic dermatitis), allergic encephalomyelitis, myocarditis, relapsing polychondritis, rheumatic heart disease, glomerulonephritis (e.g., IgA nephropathy), Multiple Sclerosis, Neuritis, Uveitis Ophthalmia, Polyendocrinopathies, Purpura (e.g., Henloch-Scoenlein purpura), Reiter's Disease, Stiff-Man Syndrome, Autoimmune Pulmonary Inflammation, Guillain-Barre Syndrome, insulin dependent diabetes mellitis, juvenile onset diabetes, and autoimmune inflammatory eye, autoimmune thyroiditis, hypothyroidism (i.e., Hashimoto's thyroiditis), systemic lupus erythematosus, Goodpasture's syndrome, Pemphigus, Receptor autoimmunities such as, for example, (a) Graves' Disease, (b) Myasthenia Gravis, and (c) insulin resistance, autoimmune hemolytic anemia, autoimmune thrombocytopenic purpura, rheumatoid arthritis, gluten-sensitive enteropathy, dense deposit disease, scleroderma with anti-collagen antibodies, mixed connective tissue disease, polymyositis/dermatomyositis, pernicious anemia (Addison's disease), idiopathic Addison's disease, infertility, glomerulonephritis such as primary glomerulonephritis and IgA nephropathy, bullous pemphigoid, Sjogren's syndrome, diabetes millitus, and adrenergic drug resistance (including adrenergic drug resistance with asthma or cystic fibrosis), chronic active hepatitis, primary biliary cirrhosis, other endocrine gland failure, vitiligo, vasculitis, post-MI, cardiotomy syndrome, urticaria, atopic dermatitis, asthma, inflammatory myopathies, and other inflammatory, granulamatous, degenerative, atrophic disorders, and other disorders such as inflammatory skin diseases including psoriasis and sclerosis, inflammatory bowel diseases (such as Crohn's disease and ulcerative colitis), respiratory distress syndrome (including adult respiratory distress syndrome, ARDS), meningitis, encephalitis, colitis, allergic conditions such as eczema and other conditions involving infiltration of T cells and chronic inflammatory responses, atherosclerosis, leukocyte adhesion deficiency, Raynaud's syndrome, and immune responses associated with acute and delayed hypersensitivity mediated by cytokines and T-lymphocytes typically found in sarcoidosis, granulomatosis and diseases involving leukocyte diapedesis, central nervous system (CNS) inflammatory disorder, multiple organ injury syndrome, antigen-antibody complex mediated diseases, anti-glomerular basement membrane disease, Lambert-Eaton myasthenic syndrome, Beheet disease, giant cell arteritis, immune complex nephritis, IgA nephropathy, IgM polyneuropathies or autoimmune thrombocytopenia etc.

According to the invention, use of the present antibody preparations can reduce or eliminate the need for immunosuppression drugs, for example in processes designed to induce immunological tolerance to a particular antigen or antigen bearing cell, tissue or organ. T-cell and or monocytes depletion or neutralization can be helpful in facilitating a state of immune tolerance such as that desired in autoimmune diseases or after organ, cell or tissue transplantation.

As shown herein, the antibody preparations of the invention deplete cellular populations of the types of cells responsible for adverse immunological reactions. Hence, by using the compositions and methods of the invention, patients have a reduced need for immunosuppression drugs.

### Pharmaceutical Compositions

Further in accordance with the present invention, the immunoglobulin preparation or composition can be formulated with a pharmaceutically acceptable carrier and/or an inert diluent. Similarly, the antigenic formulations of the invention, which include a mixture of activated lymphocytes and activated monocytes, can also be formulated with a pharmaceutically acceptable carrier and/or inert diluent for administration to generate the immunoglobulins of the invention.

The carrier should be assimilable or edible and includes liquid, semi-solid (e.g., pastes), or solid carriers. Except insofar as any conventional media, agent, diluent or carrier is detrimental to the recipient or to the therapeutic effectiveness of an immunoglobulin or antigenic preparation contained therein, its use is appropriate in an administrable composition for use in practicing the methods of the present invention. Examples of carriers or diluents include fats, oils, water, saline solutions, lipids, liposomes, resins, binders, fillers and the like, or combinations thereof.

In accordance with the present invention, the immunoglobulin or antigenic composition is combined with the carrier in any convenient and practical manner, for example, by solution, suspension, emulsification, admixture, encapsulation, absorption and the like. Such procedures are routine for those skilled in the art.

In some embodiments, the immunoglobulin or antigenic composition of the invention is in a suitable liquid carrier. The liquid carrier can include, for example, water, saline and the like, with salts, sugars, or stabilizing agents.

In other embodiment, an immunoglobulin composition of the invention is formulated as a powder. Such a powder can be combined or mixed thoroughly with a semi-solid or solid carrier. The mixing can be carried out in any convenient manner such as grinding. Stabilizing agents can be also added in the mixing process in order to protect the immunoglobulin composition from loss of therapeutic activity through, e.g., denaturation in the stomach. Examples of stabilizers for include buffers, antagonists to the secretion of stomach acids, amino acids such as glycine and lysine, carbohydrates such as dextrose, mannose, galactose, fructose, lactose, sucrose, maltose, sorbitol, mannitol, etc., proteolytic enzyme inhibitors, and the like.

Further, an immunoglobulin composition can be formulated into hard or soft shell gelatin capsules, tablets, or pills. More preferably, gelatin capsules, tablets, or pills are enterically coated. Enteric coatings prevent denaturation of the immunoglobulin composition in the stomach or upper bowel where the pH is acidic. See, e.g., U.S. Pat. No. 5,629,001. Upon reaching the small intestines, the basic pH therein dissolves the coating and permits the immunoglobulin composition to be released and absorbed by specialized cells, e.g., epithelial enterocytes and Peyer's patch M cells.

Additional formulations which are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

In other embodiments, one may use eye drops, nasal solutions or sprays, aerosols or inhalants in the present invention. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in preferred embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation.

Upon formulation, solutions are administered in a manner compatible with the dosage formulation and in such amount as is therapeutically effective to result in an improvement or remediation of signs and/or symptoms. The formulations can be administered in a variety of dosage forms such as ingestible solutions, drug release capsules and the like. Some variation in dosage can occur depending on the condition of the subject being treated. One of skill in the art can readily determine the appropriate dose for the individual subject. Moreover, for human administration, preparations generally meet sterility, pyrogenicity, general safety and purity standards as required by FDA Office of Biologics standards.

Methods for intravenous and subcutaneous administration of immunoglobulins are available in the art. For example, intravenous or subcutaneous administration of immunoglobulins is commonly used in the treatment of immunodeficient patients, patients with idiopathic thrombocytopenic purpura, and patients with a variety of autoimmune diseases.

Kits for treating or preventing inappropriate or undesirable immune responses in a patient are also provided. These kits can include an immunoglobulin preparation of the invention and instructions for administered the immunoglobulin preparation. In another embodiment, the invention provides a lit for generating immunoglobulin preparations of the invention. This kit includes an antigenic formulation having a mixture of activated lymphocytes and activated monocytes, as well as instructions for immunizing an animal with the antigenic formulation.

The invention will be further described by reference to the following detailed examples, which are given for illustration of the invention, and are not intended to be limiting thereof.

### EXAMPLE 1

### Antibodies from Animals Immunized with Activated Leukocytes Can Inhibit Immune Cell Growth and Deplete Blood Samples of Immune Cells

This Example illustrates that administration of activated lymphocytes and monocytes to animals leads to antibody production. Antibodies isolated from such animals can inhibit immune cell growth and deplete blood samples of white blood cells, most specifically, lymphocytes and monocytes.

Preparation of human leukocytes: Human Peripheral Mononuclear Blood cells (PBMC) were isolated from fresh blood by centrifugation. Purified lymphocytes and monocytes were isolated by negative selection using monoclonal antibodies against specific cell surface molecules.

Activation of human leukocytes: Human lymphocytes were activated by incubation of lymphocytes with Dynabeads (Dynal ASA, Oslo, Norway) coated with anti CD3 (OKT3) and anti-CD28 (clone 9.3) monoclonal antibody. The ratio of lymphocyte and bead was 1:4. The incubation was carried out for 18 hours. Cells were collected and washed twice with cell culture medium. Cells were then diluted with sterile normal saline.

Activation of human monocytes: Purified human monocytes were diluted with cell culture medium RPMI-1640 supplemented 10% FCS to 5×10⁶/ml and then incubated with recombinant human IFN-γ at 20-200 ng/ml for 16 hours. Cells were collected after stimulation, washed twice with cell culture medium and then diluted with sterile normal saline.

Immunization of rabbits: The rabbits were immunized with activated lymphocyte and monocytes for the initial and all subsequent boosts. Initial immunization was carried out with intradermal injections of single cell suspension of activated lymphocyte and monocytes (antigens) emulsified with complete Freund's adjuvant (1:1/volume). The subsequent boosts were given with cells suspended in 5 ml sterile normal saline via intravenous infusion. The total volume of the first immunization was no greater than one ml and intradermal injections were performed in multiple sites up to at least 10 sites on the back of rabbits with a 25 g 1-inch needle. 100×10⁶ activated lymphocytes and 100×10⁶ activated monocytes were used for initial immunization. 50-100×10⁶ activated lymphocyte and monocytes were used for the subsequent boosts.

Immunization schedule:
Day 0—initial immunization with activated lymphocytes and monocytes.
Day 21—booster immunization intravenously with activated lymphocyte and monocytes via marginal ear vein.
Day 28—booster immunization intravenously with activated lymphocyte and monocytes via marginal ear vein.
Day 29—booster immunization intravenously with activated lymphocyte and monocytes via marginal ear vein.
Day 30—booster immunization intravenously with activated lymphocyte and monocytes via marginal ear vein.
Day 35—booster immunization intravenously with activated lymphocyte and monocytes via marginal ear vein.
Day 56—booster immunization intravenously with activated lymphocyte and monocytes via marginal ear vein.

Blood collection: Rabbit blood was collected one week after day 35-booster for three weeks followed by day 56-booster. A terminal bleed was carried out under anesthesia one week after the final booster immunization.

Purification of polyclonal antibody: Polyclonal antibodies were purified with Protein G Sepharose 4 Fast Flow. (Amersham Biosciences) according to standard protocols. Rabbit anti-lymphocyte globulin (RALG) was therefore obtained as an immunoglobulin preparation prepared from serum after the animal was immunized with human activated lymphocytes and monocytes.

Mixed lymphocytes reaction: A mixed lymphocytes reaction was performed using fresh human peripheral blood mononuclear cell responders and gamma-irradiated allogeneic peripheral blood mononuclear cell stimulators. Responders and stimulators were co-incubated for 5 days with varying amounts of polyclonal IgG antibodies from rabbits immunized with either resting lymphocytes and monocytes (rabbit-1) or a combination of activated monocytes and lymphocytes (rabbit-2). The responder and stimulator cells were then pulsed with ³H-thymidine during final 24 hours. Cells were harvested onto pressed fiberglass and the uptake of ³H-thymidine by proliferating cells was measured by beta liquid scintillation counter. The results were expressed as cpm (count per minute). A xenogeneic mixed lymphocyte-endothelial cell reaction was similarly performed using fresh human lymphocytes as responders and gamma-irradiated xenogeneic porcine endothelial cells as stimulators.

### Ex Vivo Leukocyte Depletion by Rabbit Anti-Human Leukocyte

Polyclonal Antibody: Fresh blood samples collected with EDTA were incubated with rabbit anti-human leukocyte polyclonal antibody (200 ug/ml) at 37° C. for 60 minutes. Non-treated blood samples and samples treated with non-immunized rabbit IgG were used as controls. After incubation, the blood samples were sent to a clinical laboratory for a complete blood count (CBC).

Flow Cytometry Analysis: Purified monocytes (resting or activated) or lymphocytes (resting or activated) were diluted and incubated with rabbit anti-human polyclonal antibodies at 4° C. (on ice) for 60 minutes. Cells (1×10⁵) were then incubated with monoclonal antibodies (PE or FITC labeled) specific for human leukocyte surface markers such as CD25, CD54, CD52, CD45RO, and CD45RB at 40° C. for 30 minutes followed by two washes. Samples were analyzed by FACScan (Becton Dickinson).

### Immunization with Activated Leukocytes Generates Antibodies that Can Modulate Cell Proliferation

A mixed lymphocyte reaction was used to ascertain whether antibodies from animals immunized with activated leukocytes could stimulate or inhibit cell growth. Fresh human peripheral blood mononuclear cells were used as responder cells, whose cellular growth was to be measured, and gamma-irradiated allogeneic peripheral blood mononuclear cells were used as stimulator cells in the mixed lymphocyte reaction.

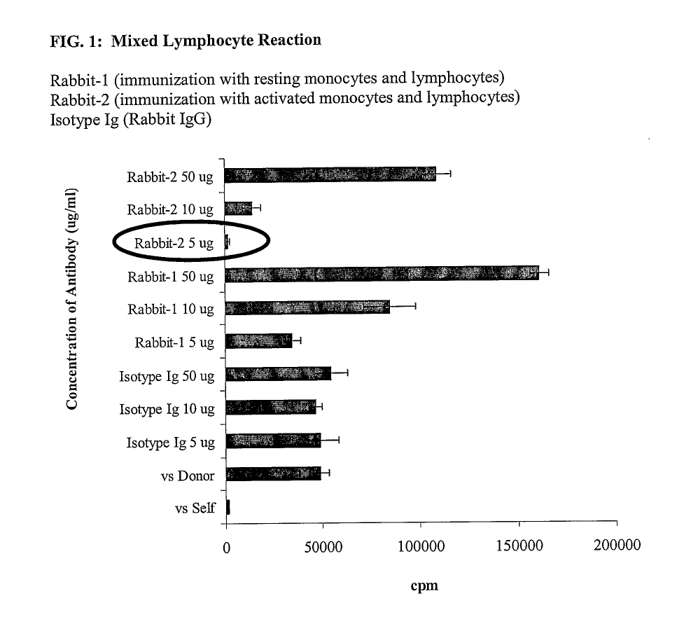

As shown in FIG. 1, small amounts of immunoglobulins isolated from a rabbit immunized with both activated monocytes and activated lymphocytes (rabbit 2) inhibit cellular proliferation of cells in a mixed lymphocyte reaction. Similar amounts of antibodies isolated from a rabbit immunized with resting leukocytes (rabbit 1) did not inhibit cellular proliferation of cells to the same extent in a mixed lymphocyte reaction. Note that high amounts of polyclonal antibodies (50 μg) can stimulate lymphocyte cell growth through binding to some cell surface markers such as CD3 and CD8. However, lower doses block lymphocyte proliferation. Efficacy at a lower dose is a desirable characteristic of biological agents used to modulate disease processes.

As controls for the mixed lymphocyte reaction assay (FIG. 1), the donor cells used to stimulate responders were separately tested (the “vs. donor” control), and irradiated autologous cells were used to simulate responders (the “vs. self” control). As shown, there is no proliferative response when stimulating with autologous cells. Donor cells give rise to a modest proliferative response similar to the response seen when using IgG from non-immunized rabbits. IgG from non-immunized rabbits did not mediate an activating or inhibitor effect indicating that the process of this invention is necessary to mediate the inhibition of the immune response in mixed lymphocyte culture.

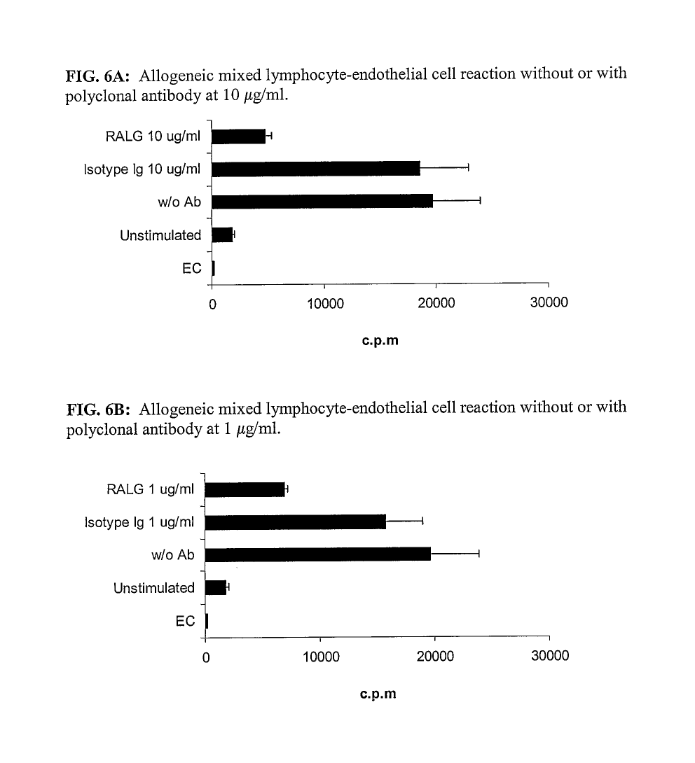

In a similar assay, FIGS. 6A and 6B shows that treatment with the rabbit anti-human leukocyte polyclonal antibodies blocks human lymphocyte proliferation stimulated by allogeneic human endothelial cells.

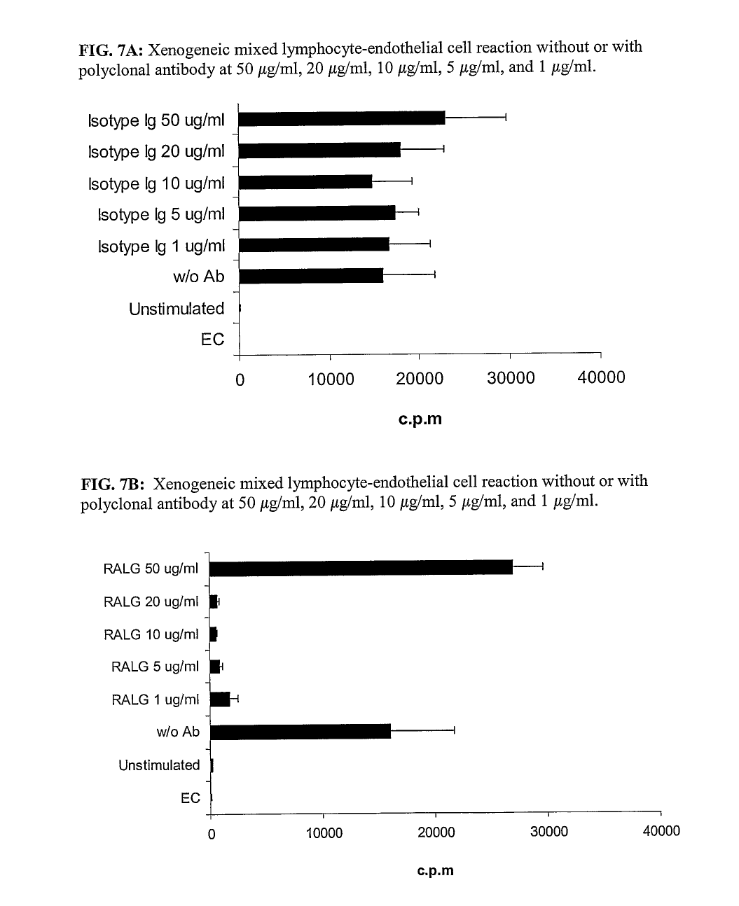

A xenogeneic mixed lymphocyte-endothelial cell reaction was also performed using fresh human lymphocytes as responders and gamma-irradiated xenogeneic porcine endothelial cells as stimulators. As shown in FIG. 7A-B, treatment with the rabbit anti-human leukocyte polyclonal antibodies block human lymphocyte proliferation in response to stimulation by xenogeneic porcine endothelial cells.

These data indicate that antibodies directed against activated lymphocytes/monocytes can bind to and modulate the growth and function of lymphocytes. In general, such antibodies inhibit lymphocyte cell growth and interactions between monocytes and lymphocytes necessary for a vigorous immune response.

### Anti-Activated Leukocyte Antibodies Deplete Blood Samples of Lymphocytes and Monocytes

Polyclonal antibodies were isolated from rabbits immunized with activated monocytes and activated lymphocytes (rabbit 2). These rabbit anti-human leukocyte polyclonal antibodies were incubated with fresh blood samples and a complete count of the cell types in the blood samples was performed.

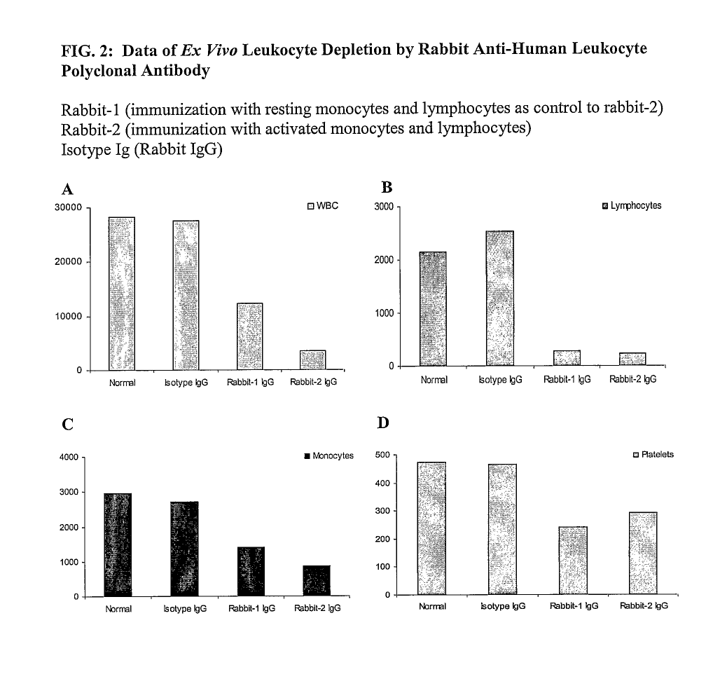

As shown in FIG. 2A, the numbers of white blood cells were lower in blood samples treated with antibodies from rabbits immunized with activated monocytes and activated lymphocytes (rabbit 2). These data indicate that the antibodies efficiently deleted monocytes and lymphocytes (both components of white blood cells), compared to blood samples treated with control antibody preparations from animals that received resting leukocytes (rabbit 1). FIGS. 2B and 2D show that polyclonal antibodies either from a rabbit immunized with activated human monocytes/lymphocytes (rabbit 2) or from a rabbit immunized with resting human leukocytes (rabbit 1) were effective at depleting blood samples of lymphocytes and platelets, but the antibodies directed against activated lymphocytes/monocytes reduced platelet counts less.

FIG. 2C illustrates that the numbers of monocytes were lower in blood samples treated with antibodies from rabbits immunized with activated monocytes and activated lymphocytes (rabbit 2), compared to blood samples treated with control antibody preparations from animals that received resting leukocytes (rabbit 1).

Thus, polyclonal antibodies from a rabbit immunized with both activated human monocytes and activated human lymphocytes (rabbit 2) were more effective for depleting blood samples of white blood cells, lymphocytes and monocytes than were polyclonal antibodies from a rabbit immunized with resting human leukocytes (rabbit 1).

### EXAMPLE 2

### Cell Markers Expressed by Activated Lymphocytes and Monocytes

Many plant substances, known collectively as lectins or phytomitogens, such as phorbol 12-myristate 13-acetate (PMA), phytohaemagglutinin (PHA), concanavalin (ConA), and lipopolyssacharide have been shown to induce blast-cell transformation and mitosis in a manner similar to alloantigens. These substances can be used to activate lymphocytes for the practice of the invention. Moreover, interferon-gamma can activate monocytes. This Example illustrates what genes (markers) are induced upon activation of lymphocytes and monocytes.

### Materials and Methods

Human lymphocytes were activated in vitro by PMA in combination with ionomycin. Briefly, lymphocytes isolated from human peripheral blood mononuclear cells are diluted with RPMI-1640 medium (Gibco, Grand Island, N.Y.) supplemented with 10% fetal calf serum (FCS) (HyClone Laboratories, Logan, Utah) to 5×10⁶ cells/ml. Cells are stimulated with PMA (3 to 30 ng/ml) and ionomycin (80 to 400 ng/ml) at 37° C. for 18 to 24 hours to induce activation. The activation was monitored by flow cytometry using antibodies directed against cell surface markers such as CD25, CD69, and CD154.

Human PBMC-derived monocytes were also activated in vitro. Purified human monocytes were diluted with cell culture medium RPMI-1640 supplemented with 10% FCS to 5×10⁶/ml and then incubated with recombinant human IFN-γ at 20-200 ng/ml for 16 hours. Cells were collected after stimulation and washed twice with cell culture medium and then diluted with sterile normal saline. The activation was monitored by flow cytometry using antibodies directed against cell surface markers such as CD40, CD80, and CD54.

### Results

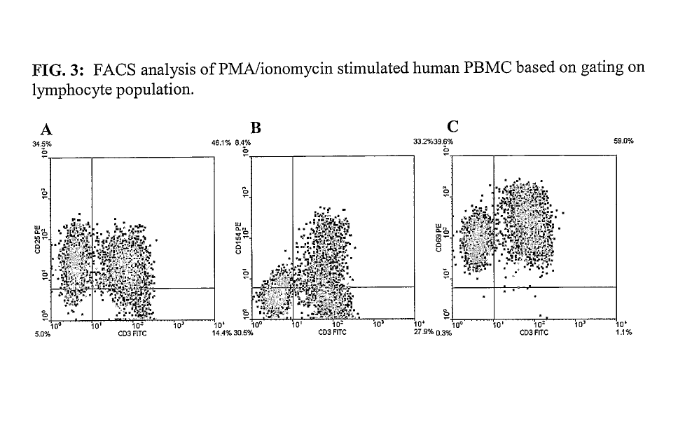

As illustrated in FIG. 3, human lymphocytes up-regulate co-stimulatory molecules such as CD154 (FIG. 3B) and cytokine receptors such as IL-2 receptor (CD25, FIG. 3A) after stimulation with PMA and ionomycin.

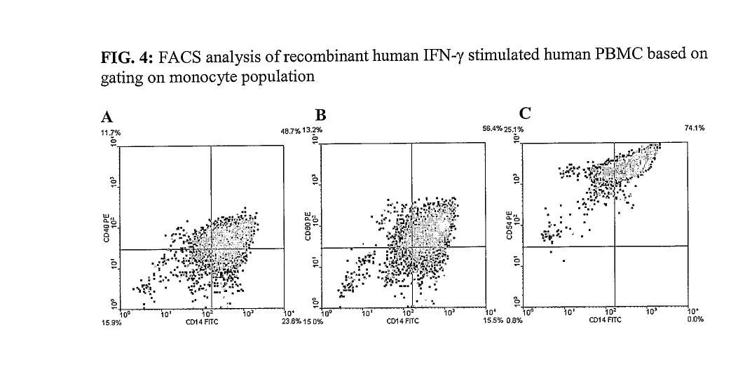

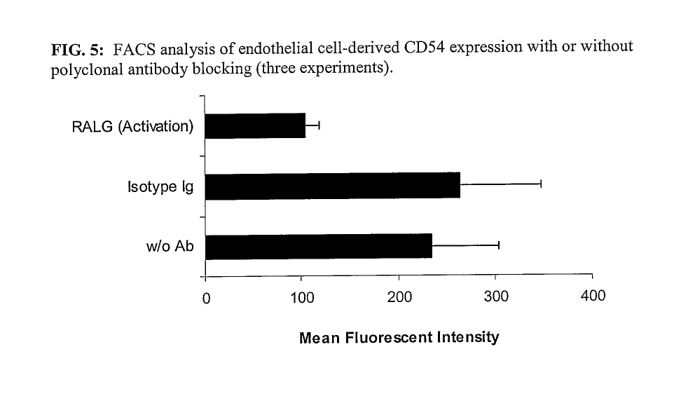

Human PBMC-derived monocytes up-regulate co-stimulatory molecules CD40 and CD80, and adhesion molecules such as CD54 (ICAM) (FIG. 4). FIG. 5 shows that polyclonal antibodies from a rabbit (RALG, rabbit 2, see Example 1) blocked endothelial-derived CD54 expression on activated human endothelial cells, as detected by FACS analysis.

### EXAMPLE 3

### Anti-Activated Leukocyte Antibodies Can Inhibit or Block Expression of Monocyte Cell Surface Markers Important for Immune Activity

Additional FACS studies were performed using activated CD14+ monocytes to ascertain whether the anti-activated lymphocyte/monocyte antibodies of the invention could modulate expression of monocyte cell markers.

Activated CD14⁺ monocytes were incubated with polyclonal antibodies from a rabbit (RALG, rabbit 2, see Example 1) and FACS analysis was performed to detect various cell markers. These studies showed that the RALG antibodies of the invention can modulate expression of human leukocyte surface markers such as CD40, CD80, CD86, CD31, CD54, CD45RO, HLA class II-DR and HLA-A, B, C (data not shown).

Quantitative FACS analysis was then performed to ascertain the degree to which marker expression was altered. Table 1 provides a summary of FACS analysis demonstrating the degree to which the RALG polyclonal antibodies blocks monocyte-derived CD11a, IFN-γ receptor-1, and CD95 expressed on activated monocytes.

TABLE 1
Antibodies from Animals Immunized with Activated
Monocytes and Lymphocytes Block Expression of a
Distinct Set of Markers on Activated Monocytes

| Surface Markers | w/o Ab | Isotype IgG | RALG |
|---|---|---|---|
| CD40 | 20 ± 14 | 22 ± 16 | 6 ± 6 |
| CD80 | 50 ± 20 | 50 ± 17 | 16 ± 10 |
| CD86 | 988 ± 167 | 1011 ± 167 | 737 ± 106 |
| HLA-DR | 280 ± 70 | 285 ± 90 | 79 ± 61 |
| HLA-ABC | 5031 ± 424 | 4931 ± 507 | 2634 ± 648 |
| CD11a | 384 ± 119 | 398 ± 91 | 198 ± 73 |
| CD31 | 602 ± 50 | 601 ± 22 | 252 ± 41 |
| CD54 | 2953 ± 763 | 2859 ± 727 | 1312 ± 615 |
| CD95 | 75 ± 41 | 78 ± 38 | 37 ± 22 |
| CD52 | 59 ± 7 | 54 ± 8 | 25 ± 9 |
| IFN-r R1 | 38 ± 13 | 37 ± 9 | 19 ± 7 |

Incubation of resting monocytes with RALG antibodies of the invention modulated expression of the markers shown in Table 1 in a different manner or to a different degree. Thus, for example, the RALG antibodies affected different markers on resting monocytes, specifically, those known to be critical in monocyte interaction with lymphocytes. Table 2 provides a summary of these results with resting monocytes, where the results are expressed as Mean Fluorescent Intensity (MFI).

TABLE 2
Blocking of monocyte surface markers following incubation
of resting monocytes with polyclonal antibody.

| Surface Markers | w/o Ab | Isotype IgG | RALG |
|---|---|---|---|
| CD86 | 94 ± 7 | 96 ± 4 | 79 ± 9 |
| HLA-DR | 41 ± 26 | 47 ± 32 | 15 ± 12 |
| CD31 | 1310 ± 385 | 1299 ± 303 | 1002 ± 191 |
| CD54 | 414 ± 292 | 407 ± 301 | 137 ± 167 |
| CD58 | 394 ± 89 | 379 ± 76 | 196 ± 117 |
| CD16 | 1395 ± 186 | 855 ± 16 | 55 ± 13 |
| CD45RB | 158 ± 29 | 167 ± 1 | 133 ± 20 |
| CD45RO | 101 ± 34 | 82 ± 9 | 85 ± 21 |
| IFN-r R1 | 151 ± 1 | 147 ± 2 | 97 ± 7 |

The results of the FACS analysis shown in Table 2 demonstrate that the RALG polyclonal antibody blocks CD16, CD31, CD54, CD58, and IFN-γ receptor-1 on resting monocytes.

### EXAMPLE 4

### Anti-Activated Leukocyte Antibodies Can Inhibit Expression of Lymphocyte Cell Surface Markers

This Example illustrates that incubation of activated peripheral blood lymphocytes with RALG polyclonal antibodies of the invention can modulate expression of various cell markers on activated lymphocytes.

RALG antibodies were obtained from a rabbit immunized with both activated human monocytes and activated human lymphocytes as described in Example 1 (rabbit 2). Activated lymphocytes were incubated with RALG polyclonal antibodies and FACS analysis was performed to detect various cell markers. These studies showed that the RALG antibodies of the invention can modulate expression of human leukocyte surface markers such as CD4, CD8, CD25, CD28, CD154, CD45RB, CD45RO and CD52 as indicated by FACS analysis (data not shown).

Further quantitative FACS analysis indicated that the RALG polyclonal antibody blocks CD25, CD28, CD154, CD45RB, CD45RO, and CD52 on activated lymphocytes. Table 3 provides a summary of these results with activated lymphocytes, where the results are expressed as Mean Fluorescent Intensity (MFI).

TABLE 3
Blocking of lymphocyte surface markers following incubation
of activated lymphocytes with RALG polyclonal antibodies

| | w/o Ab | Isotype IgG | RALG |
|---|---|---|---|
| CD25 | 100 ± 125 | 93 ± 115 | 61 ± 70 |
| CD28 | 119 ± 104 | 121 ± 92 | 71 ± 60 |
| CD154 | 49 ± 14 | 55 ± 11 | 38.5 ± 13 |
| CD45RB | 110 ± 18 | 128 ± 21 | 82 ± 27 |
| CD45RO | 104 ± 56 | 126 ± 77 | 88 ± 61 |
| CD52 | 150 ± 27 | 175 ± 38 | 126 ± 34 |

When resting peripheral blood lymphocytes were tested, expression of different cell markers was affected. Thus, incubation of resting peripheral blood lymphocytes with RALG polyclonal antibodies caused changes in the expression of human leukocyte surface markers such as CD2, CD3, CD4, CD8, CD28, CD45RB, CD45RO, CD52 and CD54 as indicated by FACS analysis (data not shown).

Quantitative FACS analysis further demonstrated that the RALG polyclonal antibody blocked CD16, CD20, and CD58 expression on resting lymphocytes. Table 4 provides a summary of these results with resting lymphocytes, where the results are expressed as Mean Fluorescent Intensity (MFI).

TABLE 4
Blocking of lymphocyte surface markers following incubation
of resting lymphocytes with RALG polyclonal antibody.

| Surface Markers | w/o Ab | Isotype IgG | RALG |
|---|---|---|---|
| CD2 | 183 ± 50 | 193 ± 69 | 38 ± 10 |
| CD3 | 861 ± 85 | 843 ± 82 | 153 ± 49 |
| CD4 | 1216 ± 288 | 1099 ± 169 | 348 ± 251 |
| CD8 | 492 ± 312 | 577 ± 399 | 107 ± 88 |
| CD16 | 976 ± 87 | 715 ± 32 | 23 ± 6 |
| CD20 | 476 ± 67 | 559 ± 103 | 264 ± 89 |
| CD28 | 122 ± 28 | 127 ± 35 | 66 ± 23 |
| CD45RB | 232 ± 33 | 249 ± 72 | 144 ± 34 |
| CD45RO | 188 ± 44 | 230 ± 21 | 116 ± 61 |
| CD58 | 91 ± 9 | 89 ± 7 | 42 ± 17 |

Further studies indicated that incubation of CD4⁺ T cells with RALG polyclonal antibodies of the invention can modulate expression of human leukocyte surface markers such as CD25, CD28, CD154, CD45RB, CD45RO, and CD52 as indicated by FACS analysis (data not shown).

### REFERENCES

Antibody-based therapies for certain diseases have been reviewed by A. Casadevall and M. D. Scharff, Clinical Infectious Diseases 1995; 150-161.
The use of antibodies for the treatment of cancer and malignancies was recently reviewed by C. Botti, A. Marinetti, S. Nerini-Molteni, and L Ferrari, Int. J. Biol. Markers 1997; 12(4):141-147; D. R. Anderson, A. Grillo-Lopez, C. Varns, and K. S. Chambers, Biochem Soc Trans 1997; 25(2):705-708; C. Renner, L. Trumper, and M. Pfreundschuh, Leukemia 1997; 11 Suppl 2:S55-59; B. Bodey, S. E. Siegel, and H. E. Kaiser, Anticancer Res 1996; 16(2):661-674.
The use of polyclonal antibody preparations for the treatment of transplant rejection was recently reviewed by N. Bonnefoy-Berard and J. P. Revillard, J Heart Lung Transplant 1996; 15(5):435-442; C. Colby, C. A. Stoukides, and T. R. Spitzer, Arm Pharmacother 1996; 30(10):1164-1174; M. J. Dugan, T. E. DeFor, M. Steinbuch, and A. H. Filipovich, Arm Hematol 1997; 75(1-2):41-46.
The use of polyclonal antibody therapies for autoimmune diseases has been described by W. Cendrowski, Boll Ist Sieroter Milan 1997; 58(4):339-343; L. K. Kastrukoff, D. R. McLean, and T. A. McPherson, Can J Neurol Sci 1978; 5(2):175-178; J. E. Walker, M. M Hoehn, and N. Kashiwagi, J Neurol Sci 1976; 29(2-4):303-309.
The depletion of fat cells using antibody preparations has been described by L. De Clercq, J. Mourot, C. Genart, V. Davidts, and C. Boone, J. Animal Sci. 1997; 75(7):1791-1797; J. T. Wright and G. J. Hausman, Obes. Res. 1995; 3(3):265-272.
The cloning of animals from cells has been described by T. Wakayama, A. C. F. Perry, M. Zuccotti, K. R. Johnson and R. Yanagachi, Nature 1998; 394:369-374; J. B. Cibelli, S. L. Stice, P. J. Golueke, J. J. Kane, J. Jerry, C. Blackwell, A. Ponce de Leon, and J. M. Robl, Science 1998; 280:1256-1258; J. B. Cibelli, S. L. Stice, P. J. Golueke, J. K. Kane, J. Jerry, C. Blackwell, F. Abel de Leon, and J. Robl, Nature Biotechnology 1998; 16:642-646; A. E. Schnieke, A. J. Kind, W. A. Ritchie, K. Mycock, A. R. Scott, M. Ritchie, I. Wilmut, A. Colman A, and K. H. Campbell, Science 1997; 278(5346):2130-2133; K. H. Campbell, J. McWhir, W. A. Ritchie, and I. Wilmut, Nature 1996; 380(6569):64-66.
Production of antibodies from transgenic animals is described in U.S. Pat. Nos. 5,814,318; 5,545,807; and 5,570,429. Homologous recombination for chimeric mammalian hosts is exemplified in U.S. Pat. No. 5,416,260. A method for introducing DNA into an embryo is described in U.S. Pat. No. 5,567,607. Maintenance and expansion of embryonic stem cells is described in U.S. Pat. No. 5,453,357.

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims. As used herein and in the appended claims, the singular forms “a,” “an,” and “the” include plural reference unless the context clearly dictates otherwise. Thus, for example, a reference to “an antibody” includes a plurality (for example, a solution of antibodies or a series of antibody preparations) of such antibodies, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed:

1. An antigenic formulation comprising a carrier and a mixture of activated mammalian lymphocytes and activated mammalian monocytes.

2. The formulation of claim 1, wherein the activated mammalian lymphocytes are human activated lymphocytes.

3. The formulation of claim 1, wherein the activated mammalian monocytes are human activated monocytes.

4. The formulation of claim 1, wherein the formulation is formulated for immunization of a mammal to generate antibodies that specifically bind to activated human lymphocytes or activated human monocytes.

5. An isolated antibody composition that binds to cell surface markers bound by an antibody preparation having ATCC Accession No. PTA-6009.

6. The composition of claim 5, wherein the antibodies are polyclonal antibodies.

7. The composition of claim 5, wherein the antibodies are human or humanized antibodies.

8. An isolated antibody obtained from a cell line having ATCC Accession No. PTA-6009.

9. A pharmaceutical composition for treating or preventing an undesirable immune response in a mammal comprising a carrier and a therapeutically effective amount of antibodies that preferentially bind to activated lymphocytes or activated monocytes.

10. The composition of claim 9, wherein the antibodies are polyclonal antibodies.

11. The composition of claim 9, wherein the antibodies are human antibodies.

12. A method of treating or preventing an undesirable immune response in a mammal comprising administering to the mammal an antibody preparation that can bind to activated mammalian lymphocytes and activated mammalian monocytes.

13. The method of claim 12, wherein the activated mammalian lymphocytes are human activated lymphocytes.

14. The method of claim 12, wherein the activated mammalian monocytes are human activated monocytes.

15. The method of claim 12, wherein the antibody preparation comprises antibodies that have a substantially human antibody sequence.

16. The method of claim 12, wherein the antibody preparation is administered more than once to the mammal.

17. The method of claim 12, wherein the mammal is a human.

18. The method of claim 12, wherein the undesirable immune response is transplant rejection, cell rejection, organ rejection, tissue rejection, arthritis, an inflammatory bowel disease, an endocrinopathy, a neurodegenerative disease, or a vascular disease.

19. The method of claim 12, wherein the undesirable immune response is rejection of allogeneic cells, rejection of allogeneic tissues, rejection of allogeneic organs, rejection of xenogeneic cells, rejection of xenogeneic tissues, rejection of xenogeneic organs, rheumatoid arthritis, psoriatic arthritis, ulcerative colitis, Crohn's disease, type 1 diabetes, Graves disease, multiple sclerosis, autistic spectrum disorder, Alzheimer's disease, amyotrophic lateral sclerosis (ALS), sclerosing cholangitis, primary biliary sclerosis, autoimmune hepatitis, systemic or discoid lupus erythematosus, Parkinson's disease, Huntington's Disease, Guillain-Barre syndrome, myasthenia gravis, chronic idiopathic demyelinating disease (CID), autoimmune hearing loss, systemic vasculitis, or atherosclerosis.

20. A method of generating antibodies that can reduce an undesirable immune response in a mammal, which comprises
(a) immunizing a host animal with an antigenic formulation comprising a carrier and a mixture of activated mammalian lymphocytes and activated mammalian monocytes; and
(b) collecting antisera from the host animal.

21. The method of claim 20, wherein the host animal generates antibodies with substantially human antibody sequences.

22. The method of claim 21, wherein the host animal has been genetically altered to produce human antibodies.

23. The method of claim 20, wherein the host animal is immunized more than once.
